(19) United States
(12) Patent Application Publication
(10) Pub. No.: US 2006/0155340 A1
(43) Pub. Date: Jul. 13, 2006

(54) Method and system to control gastrointestinal function by means of neuro-electrical coded signals

(76) Inventors: Eleanor L. Schuler, Rio Rancho, NM (US); Claude Lee, Reno, NV (US); Dennis P. Vik, Alburquerque, NM (US)

Correspondence Address: RALPH C. FRANCIS;FRANCIS LAW GROUP, 1942 EMBARCADERO, OAKLAND, CA, 94606, US

(21) Appl. No.: 11/134,767

(22) Filed: May 20, 2005

Related U.S. Application Data
(63) Continuation-in-part of application No. 11/125,480, filed on May 9, 2005.
Application No. 11/125,480 is a continuation-in-part of application No. 10/847,738, filed on May 17, 2004, now Pat. No. 6,937,903.
(60) Provisional application No. 60/572,919, filed on May 20, 2004.
(60) Provisional application No. 60/471,104, filed on May 16, 2003.

(51) Int. Cl. A61N 1/18

(52) U.S. Cl. 607/40

(57) ABSTRACT

A method to record, store and transmit waveform signals to control gastrointestinal function generally comprising capturing waveform signals that are generated in a subject's body and are operative in the control of gastrointestinal function and transmitting at least a first waveform signal to the body that is recognizable by the digestive system as a modulation signal.

### CROSS-REFERENCE TO RELATED APPLICATIONS

This application claims the benefit of U.S. Provisional Application No. 60/572,919, filed May 20, 2004, and is a continuation-in-part of U.S. application Ser. No. 11/125,480, filed May 9, 2005, which in turn is a continuation-in-part of U.S. application Ser. No. 10/847,738, filed May 17, 2004, which claims the benefit of U.S. Provisional Application Ser. No. 60/471,104, filed May 16, 2003.

### FIELD OF THE PRESENT INVENTION

The present invention relates generally to medical methods and systems for mitigating digestive system disorders. More particularly, the invention relates to a method and system for controlling gastrointestinal function by means of neuro-electrical coded signals.

### BACKGROUND OF THE INVENTION

As is well known in the art, the brain modulates (or controls) gastrointestinal function via electrical signals (i.e., action potentials or waveform signals), which are transmitted through the nervous system. The term gastrointestinal function, as used herein, means the operation of all organs and systems involved in the process of digestion, including the alimentary canal, the esophagus, the stomach, the small and large intestines, the colon, the rectum, the anus, the muscles affecting these organs, and the nervous system associated therewith.

As is known in the art, the nervous system includes two components: the central nervous system, which comprises the brain and the spinal cord, and the peripheral nervous system, which generally comprises groups of nerve cells (i.e., neurons) and peripheral nerves that lie outside the brain and spinal cord. The two systems are anatomically separate, but functionally interconnected.

As indicated, the peripheral nervous system is constructed of nerve cells (or neurons) and glial cells (or glia), which support the neurons. Operative neuron units that carry signals from the brain are referred to as “efferent” nerves. “Afferent” nerves are those that carry sensor or status information to the brain.

As is known in the art, a typical neuron includes four morphologically defined regions: (i) cell body, (ii) dendrites, (iii) axon and (iv) presynaptic terminals. The cell body (soma) is the metabolic center of the cell. The cell body contains the nucleus, which stores the genes of the cell, and the rough and smooth endoplasmic reticulum, which synthesizes the proteins of the cell.

The cell body typically includes two types of outgrowths (or processes); the dendrites and the axon. Most neurons have several dendrites; these branch out in tree-like fashion and serve as the main apparatus for receiving signals from other nerve cells.

The axon is the main conducting unit of the neuron. The axon is capable of conveying electrical signals along distances that range from as short as 0.1 mm to as long as 2 m. Many axons split into several branches, thereby conveying information to different targets.

Near the end of the axon, the axon is divided into fine branches that make contact with other neurons. The point of contact is referred to as a synapse. The cell transmitting a signal is called the presynaptic cell, and the cell receiving the signal is referred to as the postsynaptic cell. Specialized swellings on the axon's branches (i.e., presynaptic terminals) serve as the transmitting site in the presynaptic cell.

Most axons terminate near a postsynaptic neuron's dendrites. However, communication can also occur at the cell body or, less often, at the initial segment or terminal portion of the axon of the postsynaptic cell.

As with other physiologic systems, the gastrointestinal (“GI”) tract is subject to regulation by the nervous system. Indeed, the gastrointestinal tract contains over 100 million neurons, as many as the spinal cord itself.

As is well known, the gastrointestinal tract extracts nutrients from consumed food, which is then transported to the rest of the body by the blood stream. Following extraction, waste is eliminated from the body by the excretory system. Proper digestion requires the interaction of enzyme release, muscle activity and organ function, all coordinated by the nervous system.

The electrical signals transmitted along an axon to control gastrointestinal function, referred to as action potentials, are rapid and transient “all-or-none” nerve impulses. Action potentials typically have an amplitude of approximately 100 millivolts (mV) and a duration of approximately 1 msec. Action potentials are conducted along the axon, without failure or distortion, at rates in the range of approximately 1-100 meters/sec. The amplitude of the action potential remains constant throughout the axon, since the impulse is continually regenerated as it traverses the axon.

A “neurosignal” is a composite signal that includes many action potentials. The neurosignal also includes an instruction set for proper organ and/or system function. A neurosignal that controls gastrointestinal function would thus include an instruction set for the muscles of the colon and anus to perform an efficient elimination or retention of a stool bolus, including information regarding initial muscle tension, degree (or depth) of muscle movement, etc.

Neurosignals or “neuro-electrical coded signals” are thus codes that contain complete sets of information for complete organ function. As set forth in Co-Pending application Ser. No. 11/125,480, filed May 9, 2005, once these neurosignals, which are embodied in the “waveform signals” referred to herein, have been isolated, recorded, standardized and transmitted to a subject (or patient), a generated nerve-specific waveform instruction (i.e., waveform signal(s)) can be employed to control gastrointestinal function and, hence, treat a multitude of digestive system diseases and disorders, including, but not limited to, bowel (or fecal) incontinence, constipation and diarrhea.

In addition to presenting health risks, the noted disorders are embarrassing and can be socially debilitating. Persons suffering from fecal incontinence (i.e., the inability to control bowel movement) often feel shame and humiliation, and can experience social withdrawal and isolation. Although fecal incontinence is more prevalent in women and the elderly, it is not considered a normal part of aging.

A number of prior art systems and methods have been employed in an attempt to mitigate digestive system disorders. The noted systems and methods typically involve electrically stimulating portions of the gastrointestinal tract. For example, U.S. Pat. No. 6,591,137 discloses a system and method for delivering sequential stimulation to the gastrointestinal tract. Similarly, U.S. Pat. No. 5,690,691 describes a technique for pacing of the stomach and small intestine using phased stimulation with multiple electrodes. Further, U.S. Pat. No. 5,292,344 is directed to a system for delivering electrical impulses of suitable magnitude and frequency to the inner lining of the gastrointestinal tract.

There are numerous drawbacks and disadvantages associated with the noted prior art systems and methods. A major drawback is that the noted systems are typically complex and require extensive, continuous calibration. Further, the systems do not provide the type of fine control over the digestive system that is necessary to mitigate digestive system disorders, such as fecal incontinence.

An additional drawback associated with the systems and methods disclosed in the noted patents, as well as most known systems, is that the signals that are generated and transmitted to stimulate the gastrointestinal tract are “user determined” and “device determinative”. The noted “signals” are thus not related to or representative of the signals that are generated in the body and, hence, would not be operative in the control or modulation of the digestive system and, hence, gastrointestinal function.

It would thus be desirable to provide a method and system for controlling gastrointestinal function that includes means for generating and transmitting coded waveform signals to a subject's body that substantially correspond to waveform signals that are generated in the body and are operative in the control of gastrointestinal function.

It is therefore an object of the present invention to provide a method and system for controlling gastrointestinal function that overcomes the drawbacks associated with prior art methods and systems for controlling gastrointestinal function.

It is another object of the invention to provide a method and system for controlling gastrointestinal function that includes means for recording waveform signals that are generated in the body and operative in the control of gastrointestinal function.

It is another object of the invention to provide a method and system for controlling gastrointestinal function that includes means for generating digestive system waveform signals that substantially correspond to coded waveform signals that are generated in the body and are operative in the control of gastrointestinal function.

It is another object of the invention to provide a method and system for controlling gastrointestinal function that includes processing means adapted to generate a base-line gastrointestinal signal from recorded waveform signals that is representative of at least one coded waveform signal generated in the body.

It is another object of the invention to provide a method and system for controlling gastrointestinal function that includes processing means adapted to compare recorded digestive system waveform signals to baseline gastrointestinal signals and generate a coded waveform signal as a function of the noted comparison.

It is another object of the invention to provide a method and system for controlling gastrointestinal function that includes monitoring means for detecting digestive system disorders.

It is another object of the invention to provide a method and system for controlling gastrointestinal function that includes means for transmitting waveform signals to the body that substantially correspond to coded waveform signals that are generated in the body and are operative in the control of gastrointestinal function.

It is another object of the present invention to provide a method and system for controlling gastrointestinal function that includes means for transmitting signals directly to the nervous system in the body that substantially correspond to coded waveform signals that are generated in the body and are operative in the control of gastrointestinal function.

It is another object of the invention to provide a method and system for controlling gastrointestinal function that can be readily employed in the treatment of digestive system disorders, including, but not limited to, incontinence, constipation and diarrhea.

It is another object of the invention to provide a method and system for regulating gastrointestinal function without medication, biofeedback, neuromuscular reeducation, or surgery.

It is yet another object to provide a means of mitigating constipation and incontinence and other digestive system disorders and diseases of the lower gastrointestinal tract to augment conventional medicinal and surgical therapy.

### SUMMARY OF THE INVENTION

In accordance with the above objects and those that will be mentioned and will become apparent below, the method to control gastrointestinal function (in one embodiment) generally comprises (i) capturing coded waveform signals that are generated in a subject's body and are operative in the control of gastrointestinal function and (ii) transmitting at least a first waveform signal to the body that is recognizable by the digestive system as a modulation signal.

In one embodiment of the invention, the first waveform signal includes at least a second waveform signal that substantially corresponds to at least one of the captured waveform signals and is operative in the control of gastrointestinal function.

Preferably, the first waveform signal is transmitted to the subject's nervous system.

More preferably, the first waveform signal is transmitted to the pudendal nerve, the myenteric plexus, the rectal plexus, the hypogastric plexi, the intermesenteric plexus, the mesenteric ganglion/plexus, the rectal nerve, the splanchnic nerve, the lumbar chain ganglia (L-1 to L-3), the sacral plexus (S-2 to S-4) or the inferior rectal nerve.

In one embodiment of the invention, the step of transmitting a first waveform signal is adapted to control a subject's anal sphincters.

In another embodiment of the invention, the step of transmitting a first waveform signal is adapted to mediate peristaltic contraction of the gastrointestinal tract.

In another embodiment of the invention, the method to control gastrointestinal function generally comprises (i) capturing coded waveform signals that are generated in the body and are operative in control of gastrointestinal function and (ii) storing the captured waveform signals in a storage medium, the storage medium being adapted to store the components of the captured waveform signals according to the function performed by the waveform signal components, and (iii) transmitting at least a first waveform signal to the body that substantially corresponds to at least one of the captured waveform signals and is operative in the control of gastrointestinal function.

In another embodiment of the invention, the method to control gastrointestinal function generally comprises (i) capturing a first plurality of waveform signals generated in a first subject's body that are operative in the control of gastrointestinal function, (ii) generating a base-line gastrointestinal function waveform signal from the first plurality of waveform signals, (iii) capturing a second waveform signal generated in the first subject's body that is operative in the control of gastrointestinal function, (iv) comparing the base-line waveform signal to the second waveform signal, (v) generating a third waveform signal based on the comparison of the base-line and second waveform signals, and (vi) transmitting the third waveform signal proximate to the subject's body, the third waveform signal being operative in the control of gastrointestinal function.

In one embodiment of the invention, the first plurality of waveform signals is captured from a plurality of subjects.

Preferably, the third waveform signal is transmitted to the subject's nervous system.

In accordance with a further embodiment of the invention, the method for controlling gastrointestinal function in a subject generally comprises (i) capturing coded waveform signals that are generated in the body and are operative in control of gastrointestinal function, (ii) monitoring the subject's digestive system and providing at least one digestive system status signal indicative of the status of the digestive system, (iii) storing the captured waveform signals and digestive system status signals in a storage medium, and (iv) transmitting at least a first waveform signal to the body that is operative in the control of gastrointestinal function in response to a digestive system status signal or component of a captured waveform signal that is indicative of a digestive system disorder.

In one embodiment of the invention, monitoring the digestive system comprises sensing stimulation of the subject's rectal stretch receptors.

In yet another embodiment, the method to control gastrointestinal function generally comprises (i) capturing a first plurality of coded waveform signals generated in a first subject's body that are operative in the control of gastrointestinal function, (ii) capturing at least a first waveform signal from the subject's body that produces an adverse digestive event, (iii) generating a confounding signal that is operative to mitigate adverse gastrointestinal function events, and (iv) transmitting the confounding waveform signal to the subject's body to mitigate the adverse digestive event.

Preferably, the noted waveform signals are transmitted to said subject's nervous system, as described above.

The system to control gastrointestinal function in accordance with one embodiment of the invention generally comprises (i) at least a first signal probe adapted to capture coded waveform signals from a subject's body, the waveform signals being representative of waveform signals naturally generated in the body and operative in the control of gastrointestinal function, (ii) a processor in communication with the signal probe and adapted to receive the waveform signals, the processor being further adapted to generate at least a first waveform signal based on the captured waveform signals, the first waveform signal being recognizable by the digestive system as a modulation signal and (iii) at least a second signal probe adapted to be in communication with the subject's body for transmitting the first waveform signal to the body to control gastrointestinal function.

Preferably, the processor includes a storage medium adapted to store the captured waveform signals.

In one embodiment, the processor is adapted to extract and store components of the captured waveform signals in the storage means according to the function performed by the signal components.

In a further embodiment, the system also includes a sensor for monitoring the subject's digestive system.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a schematic illustration of one embodiment of a gastrointestinal function control system, according to the invention;

FIG. 2 is a schematic illustration of another embodiment of a gastrointestinal function control system, according to the invention;

FIG. 3 is a schematic illustration of yet another embodiment of a gastrointestinal function control system, according to the invention; and

FIG. 4 is a schematic illustration of an embodiment of a gastrointestinal function control system that can be employed in the treatment of a digestive system disorder, according to the invention.

### DETAILED DESCRIPTION OF THE INVENTION

Before describing the present invention in detail, it is to be understood that this invention is not limited to particularly exemplified apparatus, systems, structures or methods as such may, of course, vary. Thus, although a number of apparatus, systems and methods similar or equivalent to those described herein can be used in the practice of the present invention, the preferred systems and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “a waveform signal” includes two or more such signals; reference to “a digestive system disorder” includes two or more such disorders and the like.

### Definitions

The term “nervous system”, as used herein, means and includes the central nervous system, including the spinal cord, medulla, pons, cerebellum, midbrain, diencephalon and cerebral hemisphere, and the peripheral nervous system, including the neurons and glia.

The terms “waveform” and “waveform signal”, as used herein, mean and include a composite electrical signal that is generated in the body and carried by neurons in the body, including neurocodes, neurosignals and components and segments thereof.

The term “digestion”, as used herein, means and includes all physiological processes associated with extracting nutrients from food and eliminating waste from the body.

The term “digestive system”, as used herein, means and includes, without limitation, all organs and systems involved in the process of digestion, including the alimentary canal, the esophagus, the stomach, the small intestine, the colon, the rectum, the anus, the muscles affecting these organs, and the nervous system associated therewith.

The term “gastrointestinal function”, as used herein, means and includes, the operation of all of the organs and structures of the digestive system that are involved in the process of digestion.

The term “target zone”, as used herein, means and includes, without limitation, a region of the body, such as the colonic and anal structures, whereon the application of electrical signals can induce the desired neural control without the direct application (or conduction) of the signals to a target nerve.

The terms “patient” and “subject”, as used herein, mean and include humans and animals.

The term “plexus”, as used herein, means and includes a branching or tangle of nerve fibers outside the central nervous system.

The term “ganglion”, as used herein, means and includes a group or groups of nerve cell bodies located outside the central nervous system.

The term “incontinence”, as used herein, means and includes the inability to control bowel movement.

The terms “digestive system disorder”, “digestive disorder”, “digestive system distress” and “adverse digestive system event”, as used herein, mean and include any dysfunction of the digestive system that impedes the digestive process, such as incontinence.

As will be readily apparent to one having ordinary skill in the art, the present invention substantially reduces or eliminates the disadvantages and drawbacks associated with prior art systems and methods for controlling gastrointestinal function. As discussed in detail below, the methods of the invention include the steps of generating and transmitting at least one coded waveform signal to a subject's body that is operative in the control of gastrointestinal function. The methods (and systems) of the invention can thus be employed to mitigate a multitude of digestive system disorders, including incontinence, constipation and diarrhea.

Physiology and Function of the Gastrointestinal Tract

The digestion process begins with the mechanical breakdown of the food in the mouth though the process of mastication. Enzymatic breakdown is also commenced by salivary amylase from saliva secreted in the mouth. The vagus nerve bundle, which contains both afferent and efferent pathways, conducts neurosignals from the medulla oblongata to direct aspects of the digestive process, including the secretion of digestive chemicals and the operation of the salivary glands.

After the mechanical breakdown of the food, the food travels down the esophagus to the stomach and into the duodenum of the small intestine. Pancreatic enzymes, including chymotrypsin and trypsin, and bile continue the breakdown process. Acting as an endocrine gland, the pancreas also secretes three hormones, glucagon, somatostatin and insulin, to manage the level of glucose in response to neurosignals from the medulla oblongata.

Normally, food and digestive wastes are propelled down the gastrointestinal tract smoothly and continuously through a series of peristaltic contractions. The contractions move the waste through the small intestine, the large intestine (or colon), the rectum and finally the anal canal. This process is mediated by the gastrocolic reflex, approximately one to five times per day.

Most contractions are of the segmenting type. The frequency of the contractions is increased in the descending and sigmoid colon.

The large intestine is innervated by the lumbar splanchnic nerve and inferior mesenteric ganglion. Motor excitation is cholinergically mediated and motor inhibition is mediated by vasoactive intestinal peptide neurons.

The sigmoid colon, which forms an S-shaped loop, is located between the descending colon and rectum. The muscularis propria, which consists of outer longitudinal and inner circular muscles of the colon, enables peristalsis. This structure stores feces prior to defecation and extends from the pelvic brim to the third segment of the sacrum.

The muscularis propria receives sympathetic innervation from the lumbar (L1-L3) chain ganglia of the sympathetic trunk and the superior hypergastric plexus. Parasympathetic innervation is provided from the pelvic splanchnic nerves and sacral plexus (S2-S4).

The beginning of the rectum is indicated by the termination of the taeniae coli muscles in the sigmoid colon, which is proximal the rectosigmoid junction. The rectum descends down the sacro-coccygeal concavity (as the sacral flexure) and joins the anal canal at the anorectal junction. Three transverse folds create the superior, middle, and inferior rectal valves above the lower dilated portion, which is known as the rectal ampulla. The rectum is innervated by the hypogastric plexus, mesenteric ganglion/plexus, splanchnic, rectal, and intermesenteric plexus.

The anal canal constitutes the final portion of the alimentary tract. This structure begins at the anorectal junction and contains only circular muscle. The internal anal sphincter surrounds the anorectal junction and is a thickening of the smooth rectal circular muscle. The external anal sphincter is composed of striated muscle and surrounds the entire anal canal.

The pubocococcygeal fibers of the levator ani muscle join with the smooth longitudinal muscle of the rectum to form the conjoint longitudinal coat for the anal canal between the internal and external anal sphincters. The levator ani is composed of the puborectalis, puboccygeus, and iliococcygeus muscles and is innervated by the inferior rectal nerve and the inferior hypogastric plexus.

The rectum is usually empty, but fills intermittently after segmental contractions of the sigmoid colon. The accumulation of feces in the rectum results in a distention of the rectal ampulla, which stimulates the rectal stretch receptors that signal the myenteric plexus. The sensory neurons are able to distinguish between solid, liquid, or gas.

Defecation is a complex process integrating anal sphincter coordination, the anorectal angle, rectal compliance, anal sensitivity, and stool composition. Compliance is the ability to stretch and accommodate a bolus of feces without automatic evacuation. Anal sphincter coordination is dependant on functional sensory and motor components of sacral nerves 2, 3 and 4 and the pudendal nerve, which innervate the internal and external anal sphincters and the puborectalis.

During elimination, intra-abdominal pressure is raised by voluntary and autonomic contraction of the quadrants lumborum, rectus abdominis, transverses abdominis, diaphragm, and internal and external obliques. The external anal sphincter and the puborectalis section of the levator ani muscles of the pelvic floor are relaxed, straightening the anorectal angle to approximately 135° from the normal angle of between 60° and 105° to facilitate stool evacuation. Rectal stretch receptors stimulate the rectosphincteric reflex, which increases peristaltic wave-like contractions and relaxes the internal anal sphincter to pass the bolus into the anal canal.

Circular muscles of the rectum contract aborally to push feces toward the anus. As the feces exits, the longitudinal muscles of the rectum and levator ani bring the canal back up, expelling the bolus, and returning the anus and rectum to their normal, tightly closed position.

Defecation is under both autonomic and voluntary control. An intact sensory awareness contracts the external anal sphincter when a person becomes aware of the urge to defecate. Anal sensitivity contributes to the feeling of rectal filling, allowing conscious contraction of the external anal sphincter until evacuation is acceptable.

Conversely, the internal anal sphincter is under autonomic control. The rectosphincteric reflex increases peristalsis and relaxes the internal and external anal sphincters and produces a sensation for the urge to defecate.

If emptying of the bowel is not convenient, the reflex can be quelled through conscious contraction of the external anal sphincter. Contraction of the external anal sphincter and pelvic floor muscles results in rectal contents being expelled back into the sigmoid colon, where the bolus is stored until defecation is suitable.

The internal sphincter eventually regains its tone due to stimulus acclimation of the distended state. The rectum can also act as a storage organ, accommodating a large volume of waste.

As can be appreciated, digestion and elimination is thus dependent on numerous muscles and nerves of the abdomen, rectum, anal canal, and pelvic floor functioning properly. Moreover, various nerves are responsible for communicating sensory information from the gastrointestinal tract and conducting signals that operate the necessary muscles and physiological systems.

For example, the medulla oblongata contributes to the autonomic control of the digestive process, sending signals along the vagus nerve bundle. Neuro-electrical signals related to gastrointestinal function have also been identified as originating in the right anterior cingulate gyrus. Other regions of the brain that may contribute include the frontal cortex, the thalamus/basal ganglia complex, and the mesiotemporal lobe.

As discussed above, one disorder that can affect the digestive system includes fecal incontinence. As those having ordinary skill in the art will recognize, incontinence has several causes. For example, large hard stools caused by constipation are not easily passed through the rectum and can stretch and weaken rectal muscles, interfering with normal functioning.

Likewise, muscle damage caused by childbirth or hemorrhoid surgery can diminish the ability to contain stool. The risk of incontinence is also increased following episiotomy or use of forceps during delivery. Further, damage to nerves following brain or spinal cord injury, stroke, habitual straining at stool passage, childbirth or other traumas, and neurological diseases, such as MS, diabetes, neuropathy and spina bifida can result in fecal incontinence.

Loss of elasticity and capacity following radiation, surgery, or Irritable Bowel Syndrome (IBS) can scar rectal walls making them stiff and less compliant and prone to liquid feces leaking around solid stool. The resulting diarrhea is more difficult to control than formed solid stool.

Additionally, incontinence due to decreased or impaired sensation can be caused by childbirth or following rectal prolapse, rectocele, or general weakness of the pelvic floor. Often, these conditions manifest after forty-five years of age. Yet other factors contributing to fecal incontinence include conduction delays in the pudendal nerves to the external sphincter, pelvic floor denervation, rectal neoplasm, dementia, laxative abuse, and congenital defects.

Using the methods and systems of the invention, discussed below, incontinence and other digestive system disorders can be effectively mitigated by transmitting waveform signals that are operative in the control of gastrointestinal function.

Control of Gastrointestinal Function

As stated, the present invention substantially reduces or eliminates the disadvantages and drawbacks associated with prior art methods and systems for controlling gastrointestinal function. In one embodiment of the invention, the system for controlling gastrointestinal function generally comprises means for recording (or capturing) coded neuro-electrical or waveform signals that are generated in the body and are operative in the control of gastrointestinal function, means for storing the recorded waveform signals, means for generating at least one signal that substantially corresponds to at least one recorded waveform signal and is operative in the control of gastrointestinal function, and means for transmitting the signal to the subject's body. In a preferred embodiment of the invention, the signal is transmitted to the subject's nervous system.

According to the invention, coded neuro-electrical signals (hereinafter referred to as “waveform signals”) can be generated and transmitted to a subject that mediate the above noted physiological processes involved in digestion and the elimination or retention of a stool bolus. Moreover, the generated waveform signals, which correspond to neurosignals generated in the body, can be delivered to nerves, organs and muscles of the digestive system to control a desired aspect of gastrointestinal function.

Methods and systems for capturing coded signals from nerves, and for storing, processing and transmitting neuro-electrical signals (or coded waveform signals) are set forth in Co-Pending U.S. patent application Ser. Nos. 11/125,480, filed May 9, 2005 and Ser. No. 10/000,005, filed Nov. 20, 2001; which are incorporated by reference herein in their entirety. The noted applications also contain representative waveform signals that are operative in the control of human or animal organ function.

According to the invention, suitable neurosignals associated with gastrointestinal function can be captured or collected from any of the nerves carrying the signals to and from the gastrointestinal tract. The pudendal nerve is thus particularly suitable for capturing the noted signals. Other suitable waveforms emanate from the medullopontine region of the brain. Yet other suitable locations for capturing or recording coded signals according to the invention include the hypogastric plexi, the intermesenteric plexus, the mesenteric ganglion/plexus, the rectal nerve, the splanchnic nerve, the lumbar chain ganglia (L-1 to L-3), the sacral plexus (S-2 to S-4) and the inferior rectal nerve.

According to the invention, the captured neurosignals are transmitted to a processor or control module. Preferably, the control module includes storage means adapted to store the captured signals. In a preferred embodiment, the control module is further adapted to store the components of the captured signals (that are extracted by the processor) in the storage means according to the function performed by the signal components.

According to the invention, the stored signals can subsequently be employed to establish base-line digestive system or gastrointestinal signals. The module can then be programmed to compare “abnormal” digestive system signals (and components thereof) captured from a subject and, as discussed below, generate a waveform signal or modified base-line gastrointestinal signal for transmission to a subject. Such modification can include, for example, increasing the amplitude of a gastrointestinal function signal, increasing the rate of the signals, etc.

According to the invention, the captured neurosignals are processed by known means and a waveform signal (i.e., neuro-electrical coded signal) that is representative of at least one captured neurosignal and is operative in the control of gastrointestinal function (i.e., recognized by the brain or digestive system as a modulation signal) is generated by the control module. The noted waveform signal is similarly stored in the storage means of the control module.

To control gastrointestinal function, the generated waveform signal is accessed from the storage means and transmitted to the subject via a transmitter (or probe).

According to the invention, the applied voltage of the waveform signal can be up to 20 volts to allow for voltage loss during the transmission of the signals. Preferably, current is maintained to less than 2 amp output.

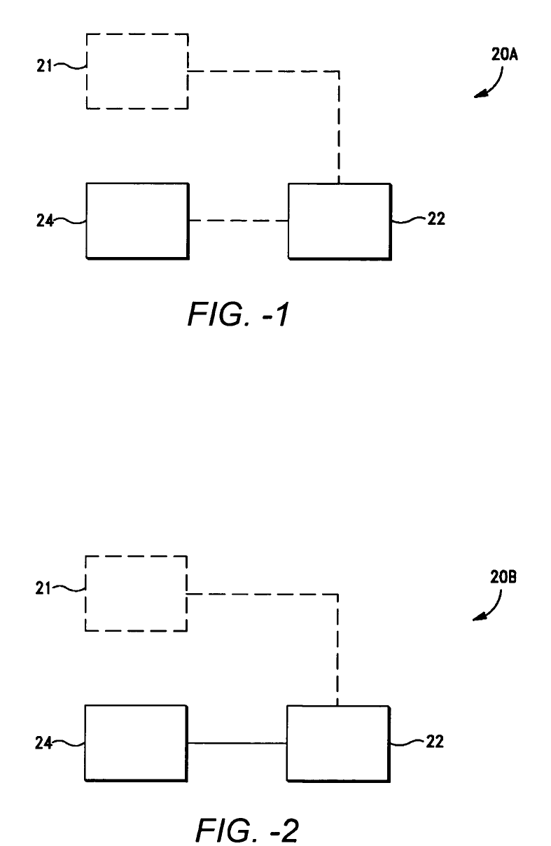

Referring now to FIG. 1, there is shown a schematic illustration of one embodiment of a gastrointestinal control system 20A of the invention. As illustrated in FIG. 1, the control system 20A includes a control module 22, which is adapted to receive neurosignals or “waveform signals” from a signal sensor (shown in phantom and designated 21) that is in communication with a subject, and at least one treatment member 24.

The treatment member 24 is adapted to communicate with the body and receives the waveform signal from the control module 22. According to the invention, the treatment member 24 can comprise an electrode, antenna, a seismic transducer, or any other suitable form of conduction attachment for transmitting coded waveform signals that regulate or operate gastrointestinal function in human or animals.

According to the invention, the treatment member 24 can be attached to appropriate nerves or digestive organ(s) via a surgical process. Such surgery can, for example, be accomplished through a “key-hole” entrance in an endoscopic procedure. If necessary, a more invasive procedure can be employed for more proper placement of the treatment member 24.

Further, if necessary, the treatment member 24 can be inserted into a body cavity, such as the nose or mouth, and can be positioned to pierce the mucinous or other membranes, whereby the member 24 is placed in close proximity to the medulla oblongata and/or pons. The waveform signals of the invention can then be sent into nerves that are in close proximity with the brain stem.

Examples of suitable transmission points for transmittal of the waveform signals of the invention by the treatment member 24 include the pudendal nerve, the hypogastric plexi, the intermesenteric plexus, the mesenteric ganglion/plexus, the rectal nerve, the splanchnic nerve, the lumbar chain ganglia (L-1 to L-3), the sacral plexus (S-2 to S-4) and the inferior rectal nerve.

As illustrated in FIG. 1, the control module 22 and treatment member 24 can be entirely separate elements, which allow system 20A to be operated remotely. According to the invention, the control module 22 can be unique, i.e., tailored to a specific operation and/or subject, or can comprise a conventional device.

Referring now to FIG. 2, there is shown a further embodiment of a control system 20B of the invention. As illustrated in FIG. 2, the system 20B is similar to system 20A shown in FIG. 1. However, in this embodiment, the control module 22 and treatment member 24 are connected.

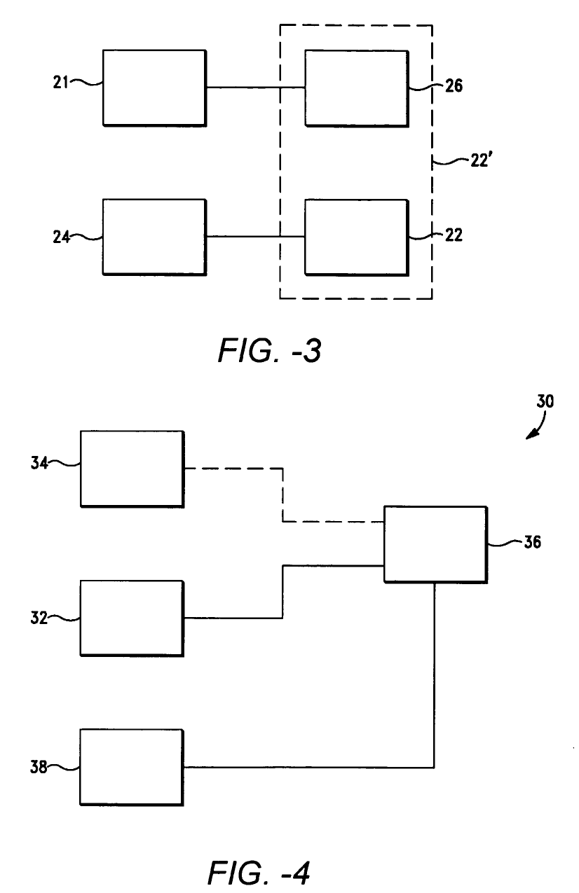

Referring now to FIG. 3, there is shown yet another embodiment of a control system 20C of the invention. As illustrated in FIG. 3, the control system 20C similarly includes a control module 22 and a treatment member 24. The system 20C further includes at least one signal sensor 21.

The system 20C also includes a processing module (or computer) 26. According to the invention, the processing module 26 can be a separate component or can be a sub-system of a control module 22′, as shown in phantom.

As indicated above, the processing module (or control module) preferably includes storage means adapted to store the captured neurosignals that are operative in the control of gastrointestinal function. In a preferred embodiment, the processing module 26 is further adapted to extract and store the components of the captured neurosignals in the storage means according to the function performed by the signal components.

According to the invention, in one embodiment of the invention, the method for controlling gastrointestinal function in a subject comprises transmitting at least one waveform signal to a subject's body that is recognizable by the digestive system as a modulation signal, the waveform signal being operative in the control of gastrointestinal function.

In another embodiment of the invention, the method for controlling gastrointestinal function in a subject includes the following steps: capturing coded waveform signals that are generated in a subject's body and are operative in the control of gastrointestinal function and (ii) transmitting at least a first waveform signal to the body that is recognizable by the digestive system as a modulation signal.

In one embodiment of the invention, the first waveform signal includes at least a second waveform signal that substantially corresponds to at least one of the captured waveform signals and is operative in the control of gastrointestinal function.

In one embodiment of the invention, the first waveform signal is transmitted to the subject's nervous system.

According to the invention, the waveform signals can be adjusted (or modulated), if necessary, prior to transmission to the subject.

In one aspect of the invention, the transmitted waveform signal is adapted to mediate contraction of the subject's anal sphincters.

In another aspect of the invention, the transmitted waveform signal is adapted to mediate peristaltic contraction of the gastrointestinal tract.

In another embodiment of the invention, the method to control gastrointestinal function generally comprises (i) capturing coded waveform signals that are generated in the body and are operative in control of gastrointestinal function and (ii) storing the captured waveform signals in a storage medium, the storage medium being adapted to store the components of the captured waveform signals according to the function performed by the signal components, and (iii) transmitting at least a first waveform signal to the body that substantially corresponds to at least one of the captured waveform signals and is operative in the control of gastrointestinal function.

In another embodiment of the invention, the method to control gastrointestinal function generally comprises (i) capturing a first plurality of waveform signals generated in a first subject's body that are operative in the control of gastrointestinal function, (ii) generating a base-line gastrointestinal waveform signal from the first plurality of waveform signals, (iii) capturing a second waveform signal generated in the first subject's body that is operative in the control of gastrointestinal function, (iv) comparing the base-line waveform signal to the second waveform signal, (v) generating a third waveform signal based on the comparison of the base-line and second waveform signals, and (vi) transmitting the third waveform signal to the body, the third waveform signal being operative in the control of gastrointestinal function.

In one embodiment of the invention, the first plurality of waveform signals is captured from a plurality of subjects.

In one embodiment of the invention, the step of transmitting the waveform signal to the subject's body is accomplished by direct conduction or transmission through unbroken skin at a zone adapted to communicate with a nerve, organ or muscle of the digestive system. Such zone will preferably approximate a position close to the nerve or nerve plexus onto which the signal is to be imposed.

In an alternate embodiment of the invention, the step of transmitting the waveform signal to the subject's body is accomplished by direct conduction via attachment of an electrode to the receiving nerve or nerve plexus. This requires a surgical intervention to physically attach the electrode to the selected target nerve.

In yet another embodiment of the invention, the step of transmitting a waveform signal to the subject's body is accomplished by transposing the waveform signal into a seismic form in a manner that allows the appropriate “nerve” to receive and obey the coded instructions of the seismic signal.

According to the invention, the control of gastrointestinal function can, in some instances, require sending waveform signals into a plurality of nerves, to provide coordinated control of gastrointestinal function to achieve the desired modulation of the digestive system.

The methods and apparatus of the invention can be effectively employed in the treatment of fecal incontinence and other digestive system ailments. Referring now to FIG. 4, there is shown one embodiment of a control system 30 that can be employed in the treatment of incontinence. As illustrated in FIG. 4, the system 30 includes at least one digestive system sensor 32 that is adapted to monitor the digestive system status of a subject and transmit at least one signal indicative of the digestive system status.

According to the invention, the digestive system status (and, hence, a digestive system disorder) can be determined by a multitude of factors, including, without limitation, muscle tension, muscle contraction, internal intestinal tract pressure, internal intestinal tract pH, etc.

As one having ordinary skill in the art will appreciate, various sensors can be employed within the scope of the invention to detect the noted factors and, hence, the onset of a digestive system disorder. According to the invention, such sensors can include temperature sensors, motion sensors and pressure sensors adapted to sense pressure within a gastrointestinal tract structure or pressure changes caused by expansion or contraction of a gastrointestinal tract structure.

The sensor can also comprise a neurosignal probe adapted to capture neurosignals transmitted to or emanating from one or more organs of the digestive system.

The system 30 further includes a processor 36, which is adapted to receive the digestive system status signal(s) from the digestive system sensor 32. The processor 36 is further adapted to receive coded waveform signals recorded by a digestive system signal probe (shown in phantom and designated 34).

In a preferred embodiment of the invention, the processor 36 includes storage means for storing the captured, coded waveform signals and digestive system status signals. The processor 36 is further adapted to extract the components of the waveform signals and store the signal components in the storage means.

In a preferred embodiment, the processor 36 is programmed to detect digestive system status signals indicative of digestive system disorders (or adverse digestive system events) and/or waveform signal components indicative of digestive system distress.

Referring to FIG. 4, the waveform signal is routed to a transmitter 38 that is adapted to be in communication with the subject's body. The transmitter 38 is adapted to transmit the waveform signal to the subject's body (in a similar manner as described above) to control the gastrointestinal function and, hence, mitigate the detected digestive system disorder.

In one embodiment of the invention, the waveform signal is preferably transmitted to the pudendal nerve to contract the anal sphincters to facilitate the retention of a stool bolus. Other suitable transmission points for the waveform signal(s) include, without limitation, the myenteric plexus, the rectal plexus, the hypogastric plexi, the intermesenteric plexus, the mesenteric ganglion/plexus, the rectal nerve, the splanchnic nerve, the lumbar chain ganglia (L-1 to L-3), the sacral plexus (S-2 to S-4) and the inferior rectal nerve.

According to the invention, a single waveform signal or a plurality of signals can be transmitted to the subject in conjunction with one another.

In accordance with a further embodiment of the invention, the method for controlling gastrointestinal function in a subject generally comprises (i) capturing coded waveform signals that are generated in the body and are operative in control of gastrointestinal function, (ii) monitoring the digestive system status of the subject and providing at least one digestive system status signal in response to an adverse digestive system event, (iii) storing the captured waveform signals and digestive system status signals in a storage medium, and (iv) transmitting at least a first waveform signal to the body that is operative in the control of gastrointestinal function in response to the digestive status signal or component of a captured waveform signal that is indicative of the adverse digestive system event.

In yet another embodiment, the method to control gastrointestinal function generally comprises (i) capturing a first plurality of coded waveform signals generated in a first subject's body that are operative in the control of gastrointestinal function, (ii) capturing at least a first waveform signal from the subject's body that produces an adverse digestive event, (iii) generating a confounding signal that is operative to mitigate the adverse digestive event, and (iv) transmitting the confounding waveform signal to the subject's body to mitigate the adverse digestive event.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

1. A method for controlling gastrointestinal function in a subject, comprising the steps of:
generating at least a first waveform signal that substantially corresponds to at least one waveform signal that is generated in said subject's body, said waveform signal being operative in the control of gastrointestinal function; and
transmitting said first waveform signal to said subject to control gastrointestinal function.

2. The method of claim 1, wherein said first waveform signal is transmitted to said subject's nervous system.

3. The method of claim 2, wherein said first waveform signal is transmitted to a location of said subject's body selected from the group consisting of the pudendal nerve, the myenteric plexus, the rectal plexus, the hypogastric plexi, the intermesenteric plexus, the mesenteric ganglion/plexus, the rectal nerve, the splanchnic nerve, the lumbar chain ganglia (L-1 to L-3), the sacral plexus (S-2 to S-4) and the inferior rectal nerve.

4. The method of claim 3, wherein said first waveform signal is adapted to control said subject's anal sphincter.

5. The method of claim 3, wherein said first waveform signal is adapted to mediate peristaltic contraction of said subject's gastrointestinal tract.

6. The method of claim 1, wherein said subject comprises a human.

7. The method of claim 1, wherein said subject comprises an animal.

8. A method for controlling gastrointestinal function, comprising the steps of:
capturing a plurality of waveform signals generated in a subject's body, said waveform signals being operative in the control of gastrointestinal function; and
transmitting at least a first waveform signal to said subject's body, said first waveform signal including at least a second waveform signal that substantially corresponds to at least one of said captured waveform signals and is operative in the control of gastrointestinal function.

9. The method of claim 8, wherein said first waveform signal is transmitted to said subject's nervous system.

10. The method of claim 8, wherein said first waveform signal is transmitted to a location of said subject's body selected from the group consisting of the pudendal nerve, the myenteric plexus, the rectal plexus, the hypogastric plexi, the intermesenteric plexus, the mesenteric ganglion/plexus, the rectal nerve, the splanchnic nerve, the lumbar chain ganglia (L-1 to L-3), the sacral plexus (S-2 to S-4) and the inferior rectal nerve.

11. The method of claim 10, wherein said first waveform signal is adapted to control said subject's anal sphincter.

12. The method of claim 10, wherein said first waveform signal is adapted to mediate peristaltic contraction of said subject's gastrointestinal tract.

13. A method for controlling gastrointestinal function, comprising the steps of:
capturing a plurality of waveform signals generated in a subject's body, said waveform signals including a plurality of signal components, said waveform signals being operative in the control of gastrointestinal function;
extracting said signal components from said captured waveform signals;
storing said captured waveform signals and said signal components in a storage medium;
generating a first waveform signal based on said captured waveform signals; and
transmitting said first waveform signal to said subject's body, said first waveform signal including at least a second waveform signal that substantially corresponds to at least one of said captured waveform signals and is operative in the control of gastrointestinal function.

14. The method of claim 13, wherein said first waveform signal is transmitted to said subject's nervous system.

15. A method for controlling gastrointestinal function, comprising the steps of:
capturing a first plurality of waveform signals generated in a first subject's body, said first plurality of waveform signals including first waveform signals that are operative in the control of gastrointestinal function;
generating a base-line gastrointestinal waveform signal from said first waveform signals;
capturing a second plurality of waveform signals generated in said first subject's body, said second plurality of waveform signals including at least a second waveform signal that is operative in the control of gastrointestinal function;
comparing said base-line gastrointestinal function waveform signal to said second waveform signal;
generating a third waveform signal based on said comparison of said base-line gastrointestinal and second waveform signals;
transmitting said third waveform signal to said first subject's body, said third waveform signal being operative in the control of gastrointestinal function.

16. The method of claim 15, wherein said step of capturing said waveform signals comprises capturing said first plurality of waveform signals from a plurality of subjects.

17. The method of claim 15, wherein said third waveform substantially corresponds to said second waveform signal.

18. The method of claim 15, wherein said third waveform substantially corresponds to said base-line gastrointestinal waveform signal.

19. The method of claim 15, wherein said third waveform signal is transmitted to a portion of said subject's nervous system capable of mediating gastrointestinal function.

20. The method of claim 19, wherein said third waveform signal is transmitted to a location of said first subject's body selected from the group consisting of the pudendal nerve, the myenteric plexus, the rectal plexus, the hypogastric plexi, the intermesenteric plexus, the mesenteric ganglion/plexus, the rectal nerve, the splanchnic nerve, the lumbar chain ganglia (L-1 to L-3), the sacral plexus (S-2 to S-4) and the inferior rectal nerve.

21. The method of claim 20, wherein said third waveform signal is adapted to control said first subject's anal sphincter.

22. The method of claim 20, wherein said third waveform signal is adapted to mediate peristaltic contraction of said first subject's gastrointestinal tract.

23. The method of claim 15, wherein said first subject comprises a human.

24. The method of claim 15, wherein said first subject comprises an animal.

25. A method for controlling gastrointestinal function, comprising the steps of:
monitoring the digestive system status of a subject and providing at least one digestive system status signal indicative of the status of said subject's digestive system;
capturing a first plurality of waveform signals generated in said subject's body, said first plurality of waveform signals including first waveform signals that are operative in the control of gastrointestinal function;
storing said digestive system status signal and said first waveform signals in a first location in a storage medium;
generating a second waveform signal based on said first waveform signals;
transmitting said second waveform signal to said subject in response to said digestive system status signal, said second waveform signal being operative in the control of gastrointestinal function.

26. The method of claim 25, wherein said second waveform signal is transmitted to said subject's nervous system.

27. The method of claim 26, wherein said second waveform signal is transmitted to a location of said subject's body selected from the group consisting of the pudendal nerve, the myenteric plexus, the rectal plexus, the hypogastric plexi, the intermesenteric plexus, the mesenteric ganglion/plexus, the rectal nerve, the splanchnic nerve, the lumbar chain ganglia (L-1 to L-3), the sacral plexus (S-2 to S-4) and the inferior rectal nerve.

28. The method of claim 27, wherein said second waveform signal is adapted to control said subject's anal sphincter.

29. The method of claim 27, wherein said second waveform signal is adapted to mediate peristaltic contraction of said subject's gastrointestinal tract.

30. A method for controlling gastrointestinal function, comprising the steps of:
monitoring a subject's digestive system and providing at least one digestive system status signal indicative of the status of said subject's digestive system;
capturing a first plurality of waveform signals generated in said subject's body, said first plurality of waveform signals including first waveform signals that are operative in the control of gastrointestinal function;
extracting the waveform signal components from said first waveform signals;
storing said digestive system status signal, said first waveform signals and said waveform signal components in a storage medium;
generating a second waveform signal based on said first waveform signals;
transmitting said second waveform signal to said subject in response to said digestive system status signal, said second waveform signal being operative in the control of gastrointestinal function.

31. The method of claim 30, wherein monitoring digestion of said subject comprises sensing stimulation of said subject's rectal stretch receptors.

32. The method of claim 30, wherein said second waveform signal is transmitted to a location of said subject's body selected from the group consisting of the pudendal nerve, the myenteric plexus, the rectal plexus, the hypogastric plexi, the intermesenteric plexus, the mesenteric ganglion/plexus, the rectal nerve, the splanchnic nerve, the lumbar chain ganglia (L-1 to L-3), the sacral plexus (S-2 to S-4) and the inferior rectal nerve.

33. The method of claim 30, wherein said second waveform signal is adapted to control said subject's anal sphincter.

34. The method of claim 30, wherein said second waveform signal is adapted to mediate peristaltic contraction of said subject's gastrointestinal tract.

35. A method for controlling gastrointestinal function, comprising the steps of:
monitoring a subject's digestive system and providing at least one digestive system status signal indicative of the status of said subject's digestive system, said status including an adverse digestive event;
capturing a first plurality of waveform signals generated in said subject's body, said first plurality of waveform signals including first waveform signals that are operative in the control of gastrointestinal function;
generating a confounding waveform signal, said confounding waveform signal being operative to mitigate said adverse digestive event in said subject's body;
transmitting said confounding waveform signal to said subject in response to a digestive system status signal indicative of said adverse digestive event.

36. The method of claim 35, wherein said adverse digestive event is selected from the group consisting of incontinence, constipation and diarrhea.

37. A system for controlling gastrointestinal function, comprising:
at least a first signal probe adapted to capture waveform signals from a subject's body, said waveform signals being representative of waveform signals naturally generated in said body and operative in the control of gastrointestinal function;
a processor in communication with said signal probe and adapted to receive said waveform signals, said processor being further adapted to generate at least a first waveform signal based on said captured waveform signals, said first waveform signal being recognizable by the digestive system as a modulation signal; and
at least a second signal probe adapted to be in communication with said subject's body for transmitting said first waveform signal to said subject's body to control gastrointestinal function.

38. The system of claim 37, wherein said processor includes a storage medium adapted to store said captured waveform signals.

39. The system of claim 37, wherein said second signal probe is adapted to transmit said first waveform signal directly to said subject by direct conduction to said subject's nervous system.

40. A system for controlling gastrointestinal function, comprising:
a digestive system sensor adapted to monitor the status of a subject's digestive system and transmit at least a first digestive system status signal indicative of the status of the subject's digestive system;
at least a first signal probe adapted to capture waveform signals from said subject's body, said waveform signals being representative of waveform signals naturally generated in said body and operative in the control of gastrointestinal function;
a processor in communication with said signal probe and adapted to receive said digestive system status signal and said waveform signals, said processor being further adapted to generate at least a first waveform signal based on said captured waveform signals, said first waveform signal being recognizable by the digestive system as a modulation signal; and
at least a second signal probe adapted to be in communication with said subject's body for transmitting said first waveform signal to said subject's body to control gastrointestinal function.

41. The system of claim 40, wherein said processor includes a storage medium adapted to store said captured waveform signals.

42. The system of claim 40, wherein said second signal probe is adapted to transmit said first waveform signal directly to said subject by direct conduction to the subject's nervous system.
